(19) United States
(12) Patent Application Publication
(10) Pub. No.: US 2005/0259891 A1
(43) Pub. Date: Nov. 24, 2005

(54) Apparatus, method, and program for producing subtraction images

(75) Inventor: Tomonari Sendai, Kanagawa-ken (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/098,611

(22) Filed: Apr. 5, 2005

(30) Foreign Application Priority Data
Apr. 5, 2004 (JP) 111124/2004
Sep. 2, 2004 (JP) 255636/2004
Mar. 25, 2005 (JP) 088130/2005

(51) Int. Cl. G21K 1/12; H05G 1/60; A61B 6/00; G01N 23/00; G06K 9/32

(52) U.S. Cl. 382/294

(57) ABSTRACT

A plurality of past cross-sectional images are reconstructed from a plurality of past tomosynthesis images obtained by radiographing a subject by tomosynthesis. A plurality of current cross-sectional images are reconstructed from a plurality of current tomosynthesis images obtained by radiographing the subject by tomosynthesis. Each of the plurality of past cross-sectional images is superimposed on a corresponding current cross-sectional image included in the plurality of current cross-sectional images for each cross-sectional plane so as to align structural elements therein. When the structural elements are aligned, a subtraction image between the corresponding past cross-sectional image and current cross-sectional image is produced.

### BACKGROUND OF THE INVENTION

1. Field of the Invention

The present invention relates to an apparatus, method, and program for producing subtraction images from images obtained by X-raying a subject.

2. Description of the Related Art

Conventionally, a temporal subtraction technique has been proposed to monitor a temporal change in a diseased area of a patient, pictured in a radiographic image. In the temporal subtraction technique, a subtraction image between temporally sequential radiographic images is produced, and a region of the radiographic image, at which the temporal change is present, is emphasized so that radiologists can easily observe the change. It is a diagnosis supporting technique for supporting the radiologists by enabling them to observe the produced subtraction image and the temporally sequential radiographic images at the same time (for example, “Digital Image Subtraction of Temporally Sequential Chest Images for Detection of Interval Change”, A. Kano, K. Doi, H. MacMahon, D. Hassell and M. L. Giger, Med. Phys. 21(3), March1994, pp. 453-461).

A method for detecting a lesion by using the temporal subtraction technique has been also proposed. In this method, a subtraction image between two images obtained by radiographing the same subject at different time is obtained. Therefore, a difference between the images is emphasized, and a lesion which has grown because of the difference in time of photography is detected (for example, Japanese Unexamined Patent Publication No. 2002-158923).

However, if the same subject is photographed at different times, the posture of the subject may be different between images obtained by radiographing. Particularly, when the chest of a human body is photographed to obtain radiographic images, the standing position or direction of a patient (subject) may change during photography. The patient may also lean forward or backward during photography. Therefore, when two two-dimensional transmitted images obtained at different times by photography are compared with each other, bone parts such as ribs and soft parts such as blood vessels and tracheas may have moved to different directions from each other in some cases. Meanwhile, as the technology of photography has improved recently, it has become possible to obtain three-dimensional transmitted images using CT (computed tomography) devices, MRI (magnetic resonance imaging) devices, or the like. However, since the three-dimensional transmitted images are expensive, radiologists obtain the three-dimensional transmitted images only at the first examination of a patient, and they obtain two-dimensional transmitted images, which are relatively inexpensive, at later examinations of the patient to monitor a temporal change in the diseased area of the patient. Therefore, a method has been proposed to accurately superimpose past images on current images. In this method, three-dimensional past image data which was obtained by radiographing the subject in the past is prepared in advance as the past images. When the subject was photographed recently, two-dimensional current image data was obtained as the current images. A three-dimensional affine transformation is performed on the three-dimensional past image data based on the change in the posture of the subject. After the three-dimensional affine transformation, a projection transformation is further performed on the three-dimensional past image data to project it onto a two-dimensional plane. Accordingly, two-dimensional past image data in which the posture of the subject is the same as that of the subject in the two-dimensional current image data can be obtained. Therefore, even if the posture of the subject is different between the past image and the current image, a temporally sequential change between the past image and the current image can be accurately observed (for example, U.S. Patent Application Publication No. 20030039405).

As disclosed in Japanese Unexamined Patent Publication No. 2002-158923, when there are two two-dimensional transmitted images in which the posture of the subject is different from each other, if a subtraction image is produced by superimposing one of the two-dimensional transmitted image on the other, an artifact is created in the subtraction image. The artifact is created because the two images are shifted from each other due to the change in the posture of the subject. Therefore, it is difficult to find a difference (a lesion or the like) between the two images in some cases.

However, when the posture of the subject during photography is different between two two-dimensional transmitted images, even if a subtraction image is produced by superimposing one of the two two-dimensional transmitted images on the other, structural elements in the images are aligned only to a certain extent. Therefore, it is difficult to superimpose the image on the other so that all of the corresponding structural elements of the subject are aligned at the same time.

Meanwhile, recently, it has become possible to obtain three-dimensional transmitted images using CT devices, MRI devices, or the like as the technique of photography has improved. Therefore, a method has been proposed to observe a temporally sequential change of a subject, as disclosed in U.S. Patent Application Publication No. 20030039405. In this method, an affine transformation is performed on three-dimensional past image data based on the change in the posture of the subject. Then, a projection transformation is further performed on the three-dimensional past image data to project it onto a two-dimensional plane. Accordingly, a two-dimensional transmitted image data in which the posture of the subject is the same as that of the subject in the two-dimensional current image data is obtained. The two-dimensional transmitted images are compared with each other to observe the temporally sequential change in the subject. However, when the two-dimensional transmitted images are compared with each other, it is impossible to know at which cross-sectional plane the change is present.

### SUMMARY OF THE INVENTION

In view of the foregoing circumstances, it is an object of the present invention to provide a subtraction image production apparatus, method and program for accurately detecting abnormal patterns or the like.

A subtraction image production apparatus according to the present invention is a subtraction image production apparatus comprising:
a superimposition means for superimposing a past cross-sectional image on a corresponding current cross-sectional image for each cross-sectional plane so as to align structural elements therein when there are a plurality of past cross-sectional images, which have been reconstructed from a plurality of past tomosynthesis images obtained by radiographing a subject by tomosynthesis, and a plurality of current cross-sectional images, which have been reconstructed from a plurality of current tomosynthesis images obtained by radiographing the subject by tomosynthesis; and
a subtraction image production means for producing a subtraction image between each pair of the past cross-sectional image and the current cross-sectional image, in which the structural elements have been aligned.

A subtraction image production method according to the present invention is a subtraction image production method comprising the steps of:
superimposing a past cross-sectional image on a corresponding current cross-sectional image for each cross-sectional plane so as to align structural elements therein when there are a plurality of past cross-sectional images, which have been reconstructed from a plurality of past tomosynthesis images obtained by radiographing a subject by tomosynthesis, and a plurality of current cross-sectional images, which have been reconstructed from a plurality of current tomosynthesis images obtained by radiographing the subject by tomosynthesis; and
producing a subtraction image between each pair of the past cross-sectional image and the current cross-sectional image, in which the structural elements have been aligned.

A program according to the present invention is a program for causing a computer to execute a subtraction image production method, the program comprising the procedures for:
superimposing a past cross-sectional image on a corresponding current cross-sectional image for each cross-sectional plane so as to align structural elements therein when there are a plurality of past cross-sectional images, which have been reconstructed from a plurality of past tomosynthesis images obtained by radiographing a subject by tomosynthesis, and a plurality of current cross-sectional images, which have been reconstructed from a plurality of current tomosynthesis images obtained by radiographing the subject by tomosynthesis; and
producing a subtraction image between each pair of the past cross-sectional image and the current cross-sectional image, in which the structural elements have been aligned.

The “current tomosynthesis images” may be images obtained in advance, as long as the images were obtained by radiographing a subject after the “past tomosynthesis images” were obtained by radiographing the subject.

A subtraction image production apparatus according to the present invention may be a subtraction image production apparatus further comprising:
an image analysis means for performing image analysis on at least one of the current tomosynthesis images and the current cross-sectional images; and
a reconstruction means for reconstructing the plurality of past cross-sectional images from the plurality of past tomosynthesis images at a narrower interval, and reconstructing the plurality of current cross-sectional images from the plurality of current tomosynthesis images at a narrower interval, if the image analysis means finds a lesion candidate.

The “lesion candidate” includes a region on which a final judgment must be made by radiologists because it is not clear whether the region is a lesion.

It is preferable that a subtraction image production apparatus according to the present invention further includes a low-frequency component removal means for removing a low-frequency component from each of the reconstructed cross-sectional images.

Further, the plurality of past tomosynthesis images may be a plurality of past tomosynthesis images obtained by radiographing a subject before the subject was dosed with a contrast medium. The plurality of current tomosynthesis images may be a plurality of current tomosynthesis images obtained by radiographing the subject after the subject was dosed with the contrast medium.

Further, the phase of cardiac cycle and/or the phase of respiration may be the same among the plurality of past tomosynthesis images. The subject may be photographed so that the phase of the cardiac cycle and/or the phase of respiration of the plurality of current tomosynthesis images is the same as that of the plurality of past tomosynthesis images.

A subtraction image production apparatus according to the present invention may further include a contrast medium photographed-area detection means for detecting the photographed area of a contrast medium, including pixels of which the pixel values are greater than or equal to a predetermined pixel value, in each of the subtraction images.

A subtraction image production apparatus according to the present invention may further include a contrast medium map generation means for generating a contrast medium distribution map by superimposing the photographed area of the contrast medium, obtained from each of the subtraction images, on each other.

Further, a subtraction image production apparatus according to the present invention may further include a contrast medium distribution emphasized image production means for producing a contrast medium distribution emphasized image by superimposing the contrast medium distribution map, generated by the contrast medium map generation means, on at least one of the plurality of past tomosynthesis images.

Further, a subtraction image production apparatus according to another embodiment of the present invention is a subtraction image production apparatus comprising:
a superimposition means for superimposing each cross-sectional image reconstructed from a plurality of tomosynthesis images obtained by radiographing a subject by tomosynthesis on an ordinary radiographic image obtained by radiographing the subject by ordinary radiographic photography so as to align structural elements therein, when there are the ordinary radiographic image and a plurality of cross-sectional images reconstructed from the plurality of tomosynthesis images, and one of the ordinary radiographic image and the plurality of tomosynthesis images is a past image or past images and the other is a current image or current images;
an addition image production means for producing an addition image by adding all of the cross-sectional images, in which the structural elements are aligned; and
a subtraction image production means for producing a subtraction image between the addition image and the ordinary radiographic image.

Further, a subtraction image production method according to another embodiment of the present invention is a subtraction image production method comprising the steps of:
superimposing each cross-sectional image reconstructed from a plurality of tomosynthesis images obtained by radiographing a subject by tomosynthesis on an ordinary radiographic image obtained by radiographing the subject by ordinary radiographic photography so as to align structural elements therein, when there are the ordinary radiographic image and a plurality of cross-sectional images reconstructed from the plurality of tomosynthesis images, and one of the ordinary radiographic image and the plurality of tomosynthesis images is a past image or past images and the other is a current image or current images;
producing an addition image by adding all of the cross-sectional images, in which the structural elements are aligned; and
producing a subtraction image between the addition image and the ordinary radiographic image.

A program according to another embodiment of the present invention is a program for causing a computer to execute a subtraction image production method, the program comprising the procedures for:
superimposing each cross-sectional image reconstructed from a plurality of tomosynthesis images obtained by radiographing a subject by tomosynthesis on an ordinary radiographic image obtained by radiographing the subject by ordinary radiographic photography so as to align structural elements therein, when there are the ordinary radiographic image and a plurality of cross-sectional images reconstructed from the plurality of tomosynthesis images, and one of the ordinary radiographic image and the plurality of tomosynthesis images is a past image or past images and the other is a current image or current images;
producing an addition image by adding all of the cross-sectional images, in which the structural elements are aligned; and
producing a subtraction image between the addition image and the ordinary radiographic image.

The method of “radiographing a subject by tomosynthesis” is a method for obtaining a plurality of “tomosynthesis images” by radiographing the subject while changing relative positions among a radiation source, a subject, and a detector by moving an X-ray tube or the like. The method of “radiographing the subject by ordinary radiographic photography” is a method for obtaining a single “ordinary radiographic image” by irradiating the subject with X-rays from an X-ray tube fixed at a single position.

The “current image” may be an image which has been obtained in advance, as long as the image was obtained by radiographing the subject after the “past photograph image” was obtained by radiographing the subject.

Further, the past ordinary radiographic image or the past tomosynthesis image may be an image obtained by radiographing the subject before the subject was dosed with a contrast medium. The current ordinary radiographic image or the current tomosynthesis image may be an image obtained by radiographing the subject after the subject was dosed with the contrast medium.

Further, the phase of the cardiac cycle and/or the phase of respiration among the plurality of past tomosynthesis images may be the same, and the subject may be photographed so that the phase of the cardiac cycle and/or the phase of respiration of the current ordinary radiographic image are the same as those of the plurality of past tomosynthesis images.

Further, the subject may be photographed so that the phase of the cardiac cycle and/or the phase of respiration of the plurality of current tomosynthesis radiographic images are the same as those of the past ordinary radiographic image.

In the present invention, past cross-sectional images reconstructed from radiographic images obtained by tomosynthesis are superimposed on current cross-sectional images reconstructed from radiographic images obtained by tomosynthesis so as to align structural elements therein, and a subtraction image is obtained for each cross-sectional plane. Accordingly, it is possible to know at which cross-sectional plane a detected change is present. Further, when a subtraction image between two-dimensional radiographic images is obtained, an error caused by the change in the posture of the subject is large. However, if a subtraction image between reconstructed cross-sectional images is obtained, such an error is small.

Further, when image analysis is performed on one of the tomosynthesis images and the cross-sectional images, if a lesion candidate is found, an interval for producing the cross-sectional images from the tomosynthesis image may be narrowed. Accordingly, detailed information may be obtained to observe the lesion only if the subject needs to be examined in detail.

Further, if a low-frequency component is removed from each of the reconstructed cross-sectional images, noise generated during the reconstruction can be eliminated.

Further, an error due to a change in the posture of the subject may be reduced by superimposing an ordinary radiographic image obtained by radiographing a subject by ordinary radiographic photography on each of cross-sectional images reconstructed from radiographic images obtained by radiographing the subject by tomosynthesis, producing an addition image by adding the cross-sectional images, and further producing a subtraction image between the addition image and the ordinary radiographic image.

Further, if the past tomosynthesis images are images obtained before the subject was dosed with a contrast medium, and the current tomosynthesis images are images obtained after the subject was dosed with the contrast medium, even if the density of the contrast medium is low, the area in which the contrast medium is photographed is emphasized. Further, since the subtraction image is obtained, surrounding structural elements can be removed. Therefore, the area in which the contrast medium was photographed is emphasized. Alternatively, one of the past images and the current image may be ordinary radiographic images and the other may be tomosynthesis images. In that case, a similar effect can be also achieved.

Further, when the past images and the current images were obtained by radiographing a subject so that the phase of cardiac cycle and/or the phase of respiration of the past images were the same as those of the current images, cross-sectional images can be obtained without being influenced from the cardiac movement or respiration. Therefore, even if a light pattern, such as that indicative of lung cancer is present in a chest image, the light pattern does not disappear. Hence, the chest region can be observed in detail.

Further, a contrast medium photographed area can be automatically detected in each subtraction image. Therefore, an area in which the contrast medium was photographed can be easily recognized.

Further, a contrast medium distribution map is generated by superimposing the contrast medium photographed area in each subtraction image on each other. Therefore, the distribution of the contrast medium can be recognized.

Further, when the contrast medium distribution map is superimposed on the tomosynthesis image, a contrast between the structural elements of the subject and the contrast medium becomes clear.

Note that the program of the present invention may be provided being recorded on a computer readable medium. Those who are skilled in the art would know that computer readable media are not limited to any specific type of device, and include, but are not limited to: floppy disks, CD's RAMI'S, ROM's, hard disks, magnetic tapes, and internet downloads, in which computer instructions can be stored and/or transmitted. Transmission of the computer instructions through a network or through wireless transmission means is also within the scope of this invention. Additionally, computer instructions include, but are not limited to: source, object and executable code, and can be in any language including higher level languages, assembly language, and machine language.

### BRIEF DESCRIPTION OF THE DRAWINGS

FIG. 1 is a diagram illustrating the schematic configuration of a subtraction image obtainment apparatus according a first embodiment of the present invention;

FIG. 2 is a diagram for explaining tomosynthesis photography;

FIG. 3 is a diagram for explaining a correspondence between cross-sectional images when a subtraction image is produced in the first embodiment;

FIG. 4A is a diagram illustrating gradient vectors;

FIG. 4B is a diagram illustrating gradient vectors;

FIG. 5 is a diagram illustrating the degree of convergence of gradient vectors;

FIG. 6 is a diagram for explaining an iris filter;

FIG. 7 is a conceptual diagram illustrating the support area of the iris filter;

FIG. 8 is a diagram for explaining a Mahalanobis distance;

FIG. 9 is a diagram illustrating the schematic configuration of another example of a subtraction image obtainment apparatus according the first embodiment of the present invention;

FIG. 10 is a diagram illustrating an example of noises in a low-frequency band;

FIG. 11 is a diagram illustrating the schematic configuration of a subtraction image obtainment apparatus which includes a contrast medium detection means;

FIG. 12 is a diagram illustrating the schematic configuration of a subtraction image obtainment apparatus according to a second embodiment of the present invention;

FIG. 13 is a diagram for explaining a correspondence between cross-sectional images and an ordinary radiographic image when a subtraction image is produced in the second embodiment; and

FIG. 14 is a diagram for explaining photography using scattered ray removal grids.

### DESCRIPTION OF THE PREFERRED EMBODIMENTS

A first embodiment of a subtraction image production apparatus according to the present invention will be described with reference to the attached drawings.

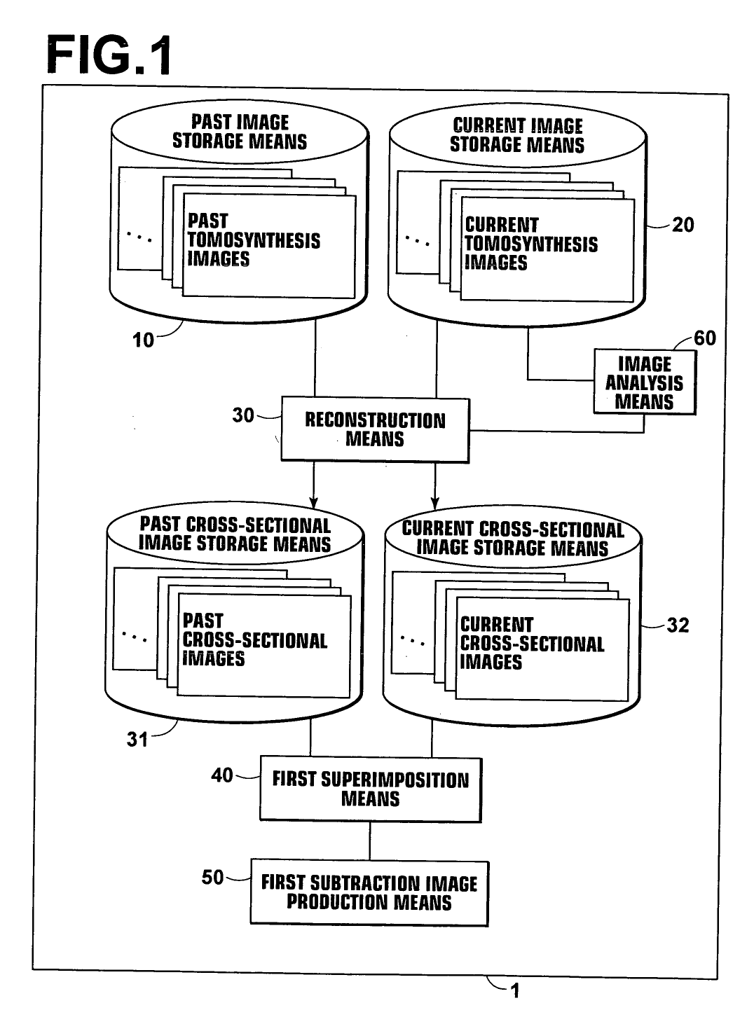

As illustrated in FIG. 1, a subtraction image production apparatus 1 according to the present invention includes a past image storage means 10 for storing past tomosynthesis images obtained by radiographing a subject by tomosynthesis. The subtraction image production apparatus 1 also includes a current image storage means 20 for storing current tomosynthesis images obtained by radiographing the subject by tomosynthesis. The subtraction image production apparatus 1 also includes a reconstruction means 30 for reconstructing cross-sectional images from the tomosynthesis images. The subtraction image production apparatus 1 also includes a first superimposition means 40 for superimposing a past cross-sectional image on a corresponding current cross-sectional image for each cross-sectional plane so as to align structural elements therein. The subtraction image production apparatus 1 also includes a first subtraction image production means 50 for producing a subtraction image between the corresponding past cross-sectional image and current cross-sectional image in which structural elements therein have been aligned. The subtraction image production apparatus 1 also includes an image analysis means 60 for performing image analysis on the radiographic images or cross-sectional images.

Tomosynthesis images are obtained by radiographing a subject by tomosynthesis to observe a diseased area of the subject in more detail. In tomosynthesis, an X-ray tube of an X-ray photography device (CR: computed radiography) is moved, and the subject is irradiated with X-rays at various angles. When the obtained tomosynthesis images are added, an image in which a desired cross-sectional plane is emphasized is obtained.

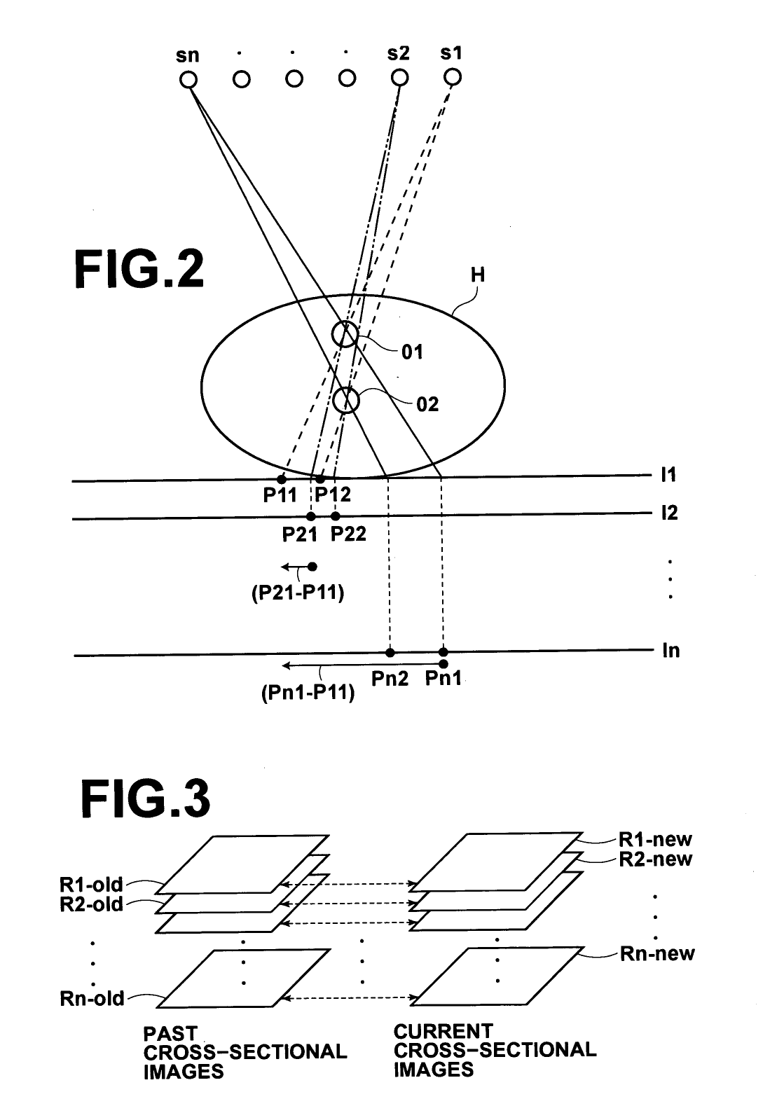

Specifically, when the subject is photographed by tomosynthesis, the X-ray tube is moved according to the characteristics of the photography device and the kind of cross-sectional images required for diagnosis. As illustrated in FIG. 2, the X-ray tube is moved parallel with a flat panel, or moved to form a circle or an oval arc. Accordingly, a subject H is photographed at various irradiation angles from each position S1, S2, . . . , and Sn, and a plurality of tomosynthesis images I1, I2, . . . , and In is obtained.

The past image storage means 10 stores a plurality of tomosynthesis images obtained in the past by radiographing the subject by tomosynthesis. The current image storage means 20 stores a plurality of tomosynthesis images, as current images, obtained by radiographing the subject after a predetermined time has passed after radiographing the subject in the past.

Specifically, the past image storage means 10 and the current image storage means 20 are provided as mass storage devices, such as hard disks, provided in a computer. The past image storage means 10 and the current image storage means 20 receive a plurality of tomosynthesis images from an X-ray photography device which has obtained the plurality of tomosynthesis images by radiographing the subject. The past image storage means 10 and the current image storage means 20 further store the plurality of tomosynthesis images. Alternatively, the chest of the subject may be photographed to obtain data, and the obtained data maybe stored in a portable storage medium such as a DVD (digital versatile disc). The stored data may be read out from the storage medium. Further, the tomosynthesis images may be stored in a file server or the like connected to a user's terminal via a network, and tomosynthesis images of the subject may be retrieved from the file server to be read.

The reconstruction means 30 reconstructs cross-sectional images from the tomosynthesis images stored in the past image storage means 10 and the current image storage means 20. The reconstruction means 30 stores the cross-sectional images reconstructed from the past tomosynthesis images in a past cross-sectional image storage means 31. The reconstruction means 30 stores the cross-sectional images reconstructed from the current tomosynthesis images in a current cross-sectional image storage means 32. The past cross-sectional image storage means 31 and the current cross-sectional image storage means 32 are mass storage devices such as a hard disk and storage media such as a DVD.

A method for reconstructing the cross-sectional images from the tomosynthesis images will be described below specifically. First, as illustrated in FIG. 2, the subject H is photographed at various irradiation angles by moving an X-ray tube to each position S1, S2, . . . , and Sn. Accordingly, tomosynthesis images I1, I2, . . . , and In are obtained. For example, if objects (01, 02) which are present at different depths are projected from a radiation source at position S1, they are projected onto the tomosynthesis image I1 at positions P11 and P12, respectively. If the objects (01, 02) are projected from the radiation source at position S2, they are projected onto the tomosynthesis image I2 at positions P21 and P22, respectively. If the objects are repeatedly projected from the radiation source at various positions S1, S2, . . . , and Sn, the object 01 is projected onto the tomosynthesis images at positions P11, P21, . . . and Pn1 as the position of the radiation source moves. The object 02 is projected onto the tomosynthesis images at the positions of P12, P22, . . . and Pn2 as the position of the radiation source moves.

If radiologists want to emphasize a cross-sectional plane on which the object 01 is present, the tomosynthesis image I2 is moved by a distance (P21-P11) between P21 and P11. Then, the tomosynthesis image I3 is moved by a distance (P31-P11) between P31 and P11, . . . , and the tomosynthesis image In is moved by a distance (Pn1-P11). Then, all the moved images are added. Accordingly, a cross-sectional image R1 in which a structural element which is present on the cross-sectional plane at the depth of the object 01 is produced. Further, if the radiologists want to emphasize a cross-sectional plane on which the object 02 is present, the tomosynthesis image I2 is moved by a distance (P22-P12) between P22 and P12. Then, the tomosynthesis image I3 is moved by a distance (P32-P12) between P32 and P12, and the tomosynthesis image In is moved by a distance (Pn2-P12) between Pn2 and P12. Then, all the moved images are added. Accordingly, a cross-sectional image R2 is produced. As described above, the structural elements in each of the tomosynthesis images I1, I2, . . . and In are aligned according to the position of a cross-sectional plane required by the radiologists, and the tomosynthesis images in which the structural elements are aligned are added. Accordingly, cross-sectional images (R1, R2, . . . , and Rn) may be produced by emphasizing structural elements which are present on the cross-sectional planes produced at desired intervals.

The first superimposition means 40 superimposes past cross-sectional images R1-old, R2-old, . . . , and Rn-old which are reconstructed from past tomosynthesis images on current cross-sectional images R1-new, R2-new, . . . , and Rn-new which are reconstructed from current tomosynthesis images so as to align structural elements therein. The first superimposition means 40 superimposes the past cross-sectional image on a corresponding current cross-sectional image between each pair of corresponding cross-sectional images (R1-old and R1-new, R2-old and R2-new, . . . , and Rn-old and Rn-new), as illustrated in FIG. 3.

When the past cross-sectional images are superimposed on the current cross-sectional images, the structural elements in the corresponding cross-sectional images should be aligned. Therefore, it is necessary to superimpose the past cross-sectional images on the current cross-sectional images so that the positions (anatomical characteristic positions) of structural elements in the past cross-sectional images are matched with those of the structural elements in the current cross-sectional images. As a method for superimposing the past cross-sectional images on the corresponding current cross-sectional images as described above, a method of performing an affine transformation or the like to generally align structural elements in two corresponding images and further performing a non-linear warp transformation (warping) by curve-fitting to locally align the structural elements in the images which have been generally aligned already, as disclosed in, for example (Japanese Unexamined Patent Publication No. 7(1995)-037074, U.S. Pat. No. 5,982,915, or the like) may be employed.

Specifically, first, two images are generally aligned by performing processing such as parallel translation, rotation, or enlargement/reduction (linear alignment using affine transformation or the like). Then, regions of interest (template regions), which are a multiplicity of small regions, are set in one of the two images which have been generally aligned. Search regions corresponding to the template regions, each of which is larger than the corresponding template region, are set in the other image. Then, regarding each pair of the template region and the corresponding search region, a sub-region (corresponding template region) is obtained in each of the search regions. The sub-region is a region in which the image is substantially the same as that of the template region. Further, a shift amount is obtained based on the corresponding positional relationship between each of the template regions in one image and a corresponding template region in the other image. The shift amount is an amount by which the image should be shifted to match each of the template regions in one image with a corresponding template region in the other image. Then, non-linear warp transformation (warping) by curve fitting (for example two-dimensional n-th polynomial, N≧2) is performed, based on the obtained shift amount, on the two images which have been generally aligned.

Further, another method has been proposed by the assignee of the present application (Japanese Unexamined Patent Publication No. 2001-325584 and Japanese Unexamined Patent Publication No. 2002-324238). In this method, images in which a particular structural element is emphasized are used. The images are aligned so that the particular structural element in one image matches with the same structural element in the other image. According to this method, when images in which bone tissues are emphasized are used, images which have been generally aligned regarding the bone tissues can be obtained.

Further, a method of locally aligning images after the images are generally aligned based on the purpose of alignment has been proposed by the assignee of the present application (Japanese Unexamined Patent Publication No. 2002-324238). In this method, if a user wants to extract a lesion which is present in a bone part, the user may select processing for emphasizing the bone part. If the user wants to extract a lesion which is present in a soft part, the user may select processing for not emphasizing the bone part. If this method of aligning the images in two steps is used, the two images can be aligned relatively well for the purpose of alignment.

The image analysis means 60 analyzes the tomosynthesis images and the cross-sectional images produced from the tomosynthesis images, and detects a lesion candidate, which is a candidate of a lesion such as a tumor.

For example, the contours of tumor patterns, which represent malignant areas, in a radiographic image or the like are substantially circular. At the same time, the tumor patterns are observed as areas including pixels of which the pixel values are higher than those of the pixels in the surrounding area in the image. The tumor patterns are hemispherical areas including concentric circles each formed by pixels having the same density value (hereinafter, the areas will be called “rounded convex regions). A gradient in the density is observed, and the density values are distributed so that the density values are highest at the peripheral circle, and they decrease toward the center of the circular area. The gradient of the density converges at the center of the tumor. Therefore, the lesion candidate such as the tumor pattern can be detected based on the degree of convergence of the gradient vectors by calculating the gradient vectors (please refer to Jun Wei, Yoshihiro Hagiwara, Akinobu Shimizu, Hidefumi Kobatake, “An Analysis on the Characteristics of a Point-Convergence Filter”, Journal of the Institute of Electrics, Information, and Communication Engineers (D-11), Vol. J84-D-II, No. 7, pp. 1289-1298, 2001, or Jun Wei, Yoshihiro Hagiwara, Hidefumi Kobatake, “A Gradient Vector Convergence Filter for Extracting Cancer Pattern Candidates”, Journal of the Institute of Electrics, Information and Communication Engineers (D-11), Vol. J83-D-II, No. 1, pp. 118-125, January 2001).

Specifically, the degree of convergence of the gradient vectors can be obtained as described below.

First, regarding all the pixels included in an image which is an object for calculation, the orientation φ of the gradient vector of image data is obtained based on the following equation (1):

$$ϕ = {tan}^{- 1} \frac{\begin{matrix}( {f}_{11} + {f}_{12} + {f}_{13} + {f}_{14} + {f}_{15} ) - \\ ( {f}_{51} + {f}_{52} + {f}_{53} + {f}_{54} + {f}_{55} )\end{matrix}}{\begin{matrix}( {f}_{15} + {f}_{25} + {f}_{35} + {f}_{45} + {f}_{55} ) - \\ ( {f}_{11} + {f}_{21} + {f}_{31} + {f}_{41} + {f}_{51} )\end{matrix}} \qquad ( 1 )$$

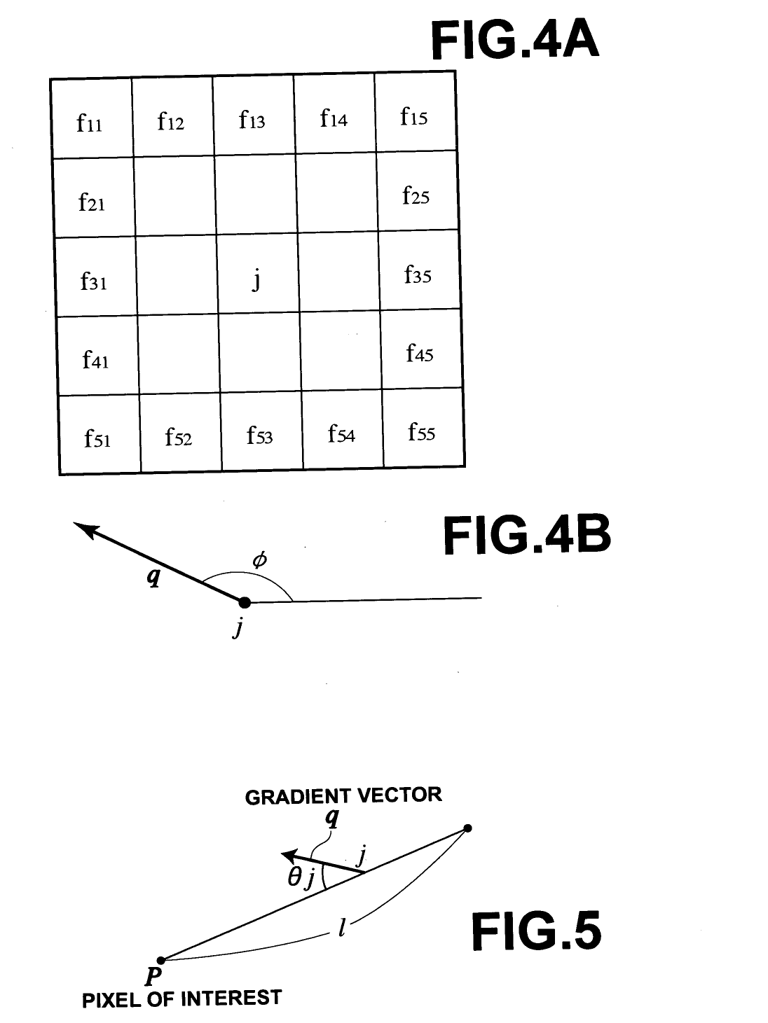

Here, values f11-f55 are pixel values (image data) corresponding to pixels on the outer circumference of a mask of 5×5 (five pixels in a vertical direction and five pixels in a horizontal direction) with the center of the mask at pixel j as illustrated in FIGS. 4A and 4B.

Then, regarding all the pixels included in the image which is the object of calculation, the degree of convergence C of the gradient vectors is calculated based on the following equation (2):

$$C = ( 1 / N ) {\sum }_{j = 1}^{N} cos {θ}_{j} \qquad ( 2 )$$

Here, value N is the number of pixels which are present in a circle with a radius of 1 with the center of the circle at the pixel of interest. Value φj is an angle formed by a straight line connecting the pixel of interest and each pixel j in the circle and a gradient vector calculated based on the above equation (1) at each pixel j (please refer to FIG. 5). Therefore, the degree of convergence C, represented by the above equation (2), is large at a pixel at which the direction of the gradient vector of each pixel j converges.

In other words, the gradient vector of each pixel j in the vicinity of the tumor pattern approximately points to the center of the tumor pattern regardless of the degree of the contrast of the tumor pattern. Therefore, a pixel at which the degree of convergence C is large is a pixel at the center of the tumor pattern. However, the degree of convergence is small in patterns of linear patterns such as blood vessels because the directions of the gradient vectors point to a certain direction in the patterns of the linear patterns. Hence, a lesion candidate such as a tumor pattern may be detected by calculating the value of the degree of convergence C for each of all the pixels included in the image with respect to a pixel of interest, and evaluating whether the value of the degree of convergence C exceeds a predetermined threshold value.

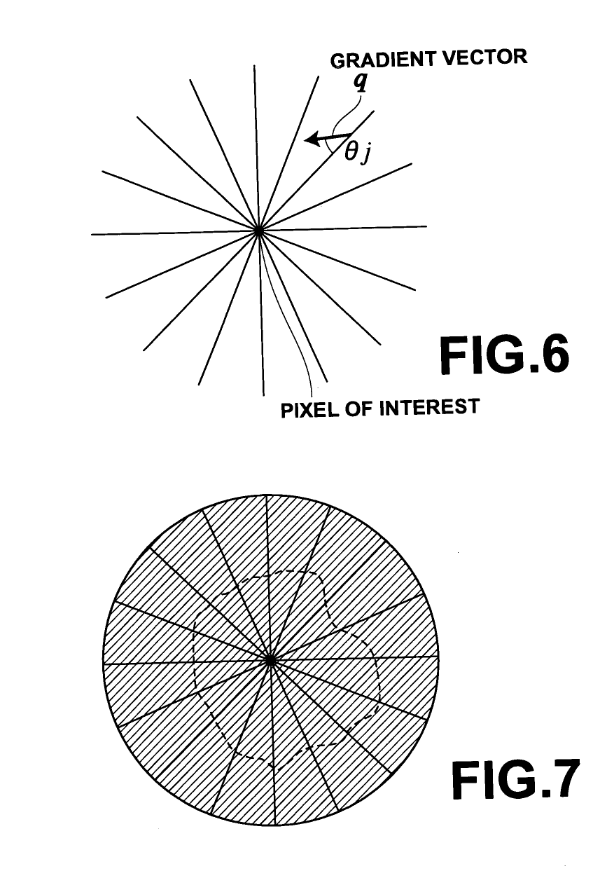

Further, as a method for evaluating the degree of convergence, there is a method using a filter which has an appropriate size and shape for detecting a tumor pattern so that the tumor pattern is detected regardless of the size and shape of the tumor. A typical example of such filter is an iris filter. The iris filter is a filter, of which the filer size changes adaptively according to the size of a tumor. As illustrated in FIG. 6, the degree of convergence is evaluated using only the pixels on radial lines formed in M number of directions with the center at the pixel of interest. The M number of directions are directions of the radial lines which are formed every 2π/M degrees (in FIG. 6, 16 directions in every 25 degrees are illustrated).

The degree of convergence of pixels on a single semi-straight line is obtained based on the equation (3):

$${C}_{J}^{0} = \begin{matrix}max \\ N1 \leq N \leq N0\end{matrix} {c}_{j} \qquad ( 3 )$$

Here, value cⱼ is the degree of convergence obtained by the equation (2). Values N₁ and N₀ are the minimum number of pixels and the maximum number of pixels, respectively, which are counted from the pixel of interest on the semi-straight lines. In this case, an output from the iris filter can be obtained based on the following equation (4):

$$C ( x , y ) = \frac{1}{M} {\sum }_{i = 0}^{M - 1} {C}_{J}^{0} \qquad ( 4 )$$

As it is apparent from the equation which defines the filter, the support area of the iris filter changes as a pixel of interest is changed to another pixel of interest. FIG. 7 is a conceptual diagram illustrating this characteristic. In FIG. 7, an output from the iris filter with respect to a pixel of interest is represented by the degree of convergence of M number of semi-straight lines. A calculation range of the filter in a single semi-straight line extends to a point (a range indicated with a dotted line) at which the maximum value is obtained by the equation (3). Therefore, the area in which the output from the filter becomes the maximum can be extracted as a lesion candidate region.

Further, a candidate region is detected using an iris filter or the like. Then, a histogram of density in the area is obtained based on the detected lesion candidate. A plurality of characteristic values such as a variance value, contrast, and angular moment are calculated based on the histogram. Further, a new evaluation value is calculated by defining each characteristic value using a predetermined weighting function. Then, a judgment is made as to whether the lesion candidate region is a malignant pattern based on the calculated evaluation value. Accordingly, only the malignant pattern may be detected as a lesion candidate (for details, please refer to Japanese Unexamined Patent Publication No. 8(1996)-294479 and Japanese Unexamined Patent Publication No. 9(1997)-167238, for example).

Besides the characteristic values described above, edge information representing the characteristic of the edge of the candidate region may be used as the characteristic values. The edge information includes a variance value, deviation, correlation value, moment, entropy, or the like. Further, a Mahalanobis distance may be used as the evaluation value. The Mahalanobis distance is represented by a value Dmi, defined by the following equation (6). The Mahalanobis distance is a distance representing variance from the center of distribution of the characteristic values of a malignant pattern or a benign pattern. The Mahalanobis distance is represented by a covariance matrix Σ.
Dmi−(\vec{x}−\vec{m}i)^{t}Σᵢ⁻¹(\vec{x}−\vec{m}i) (6)
where Σᵢ is a covariance matrix of a pattern class (if i=1, patterns of benign patterns, and if i=2, patterns of malignant patterns) wi. Therefore,

$${\sum }_{i} = ( 1 / Ni ) {\sum }_{x ∈ wi} ( {x}^{->} - {m}^{->} i ) {( {x}^{->} - {m}^{->} i )}^{t}$$
where t represents a transposed vector (row vector), and \vec{x} is a vector representation of a characteristic value x. Therefore,
   \vec{x}=(x1,x2, . . . ,xN).

Σᵢ⁻¹ is an inverse matrix of Σᵢ.

\vec{m} is an average of the pattern classes wi. Therefore,

$${m}^{->} i = ( 1 / Ni ) {\sum }_{x ∈ wi} {x}^{->}$$

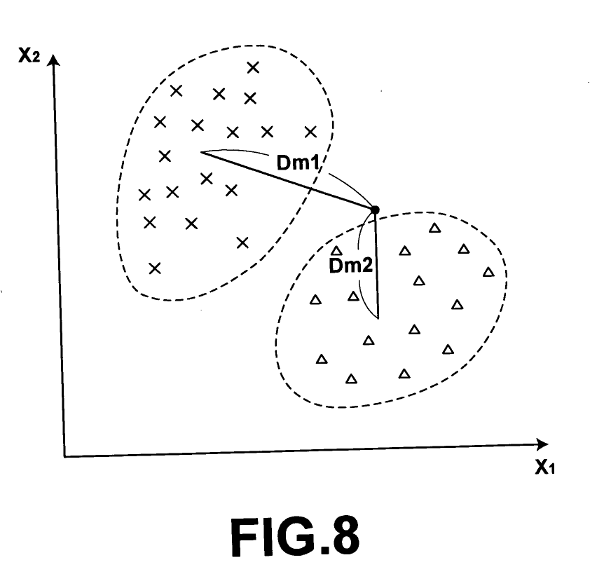

Then, a Mahalanobis distance Dm1 from a pattern class (i=1) which represents a benign pattern and a Mahalanobis distance Dm2 from a pattern class (i=2) which represents a malignant pattern are calculated based on the equation (6). Both of the pattern classes have been experimentally obtained in advance. The Mahalanobis distance Dm1 and the Mahalanobis distance Dm2 are compared with each other to judge whether the candidate region is a malignant area (refer to FIG. 8). Specifically, if the Mahalanobis distance Dm1 from the pattern class representing a benign pattern is less than the Mahalanobis distance Dm2 from the pattern class representing a malignant pattern, in other words, if Dm1<Dm2, it is judged that the candidate region is a benign pattern. If the Mahalanobis distance Dm1 from the pattern class representing a malignant pattern is less than the Mahalanobis distance Dm2 from the pattern class representing a benign pattern, in other words, if Dm1>Dm2, it is judged that the candidate region is a malignant pattern. Only the candidate region that has been judged as the malignant pattern may be detected as a lesion candidate (please refer to Japanese Unexamined Patent Publication No. 2002-074325, for example).

Here, a flow of observation of a temporal change in a predetermined region of a subject using the subtraction image apparatus according to the present invention will be described specifically. The temporal change is detected by observing cross-sectional images which have been reconstructed from tomosynthesis images.

First, while an X-ray tube of an X-ray photography device is moved sequentially from point S1 to S2, . . . , and Sn, the subject is photographed by tomosynthesis to obtain a plurality of tomosynthesis images. The obtained images are stored in the past image storage means 10.

After a predetermined time has passed, the same region of the same subject is photographed again by tomosynthesis to detect a temporal change in the lesion of the subject. A plurality of tomosynthesis images is obtained and stored in the current image storage means 20.

The reconstruction means 30 reconstructs cross-sectional images from the tomosynthesis images stored in the past image storage means 10 and the current image storage means 20. The cross-sectional images are produced at an interval of approximately 5 mm through 1 cm, for example. Cross-sectional images R1-old, R2-old, . . . , and Rn-old are reconstructed from the past tomosynthesis images, and the cross-sectional images are stored in the past cross-sectional image storage means 31. Cross-sectional images R1-new, R2-new, . . . , and Rn-new are reconstructed from the current tomosynthesis images, and the cross-sectional images are stored in the current cross-sectional image storage means 32.

Then, the first superimposition means 40 superimposes past cross-sectional images R1-old, R2-old, . . . , and Rn-old which are stored in the past cross-sectional image storage means 31 on current cross-sectional images R1-new, R2-new, . . . , and Rn-new which are stored in the current cross-sectional image storage means 32 so as to align structural elements therein. The first superimposition means 40 align the structural elements between the corresponding cross-sectional images (R1-old and R1-new, R2-old and R2-new, . . . , and Rn-old and Rn-new, for example) as illustrated in FIG. 3. Then, the first subtraction image production means 50 produces subtraction images between the images in which the structural elements have been aligned. The first subtraction image production means 50 produces the same number of subtraction images as the number of the cross-sectional images.

A temporal change on each of the cross-sectional planes can be detected by observing the obtained subtraction images. Therefore, it is possible to observe on which cross-sectional plane a lesion has changed.

If the image analysis means 60 detects a lesion candidate by performing image analysis on one of the current tomosynthesis images stored in the current image storage means 20, the cross-sectional images are produced at narrower intervals. For example, the cross-sectional images are normally produces at regular intervals of approximately 5 mm through 1 cm. However, if a lesion candidate is found, the cross-sectional images are produced at intervals of approximately 3 mm. It is required to observe the image in detail to detect a lung cancer or the like. Therefore, it is preferable that the cross-sectional images are produced at intervals of approximately 1 mm to detect a lung cancer or the like. The first subtraction image production means 50 produces a subtraction image using the cross-sectional images which are reconstructed from the tomosynthesis images at narrow intervals. Accordingly, the image can be observed in detail. Further, the image analysis may be performed on one of the current cross-sectional images stored in the current cross-sectional image storage means 32.

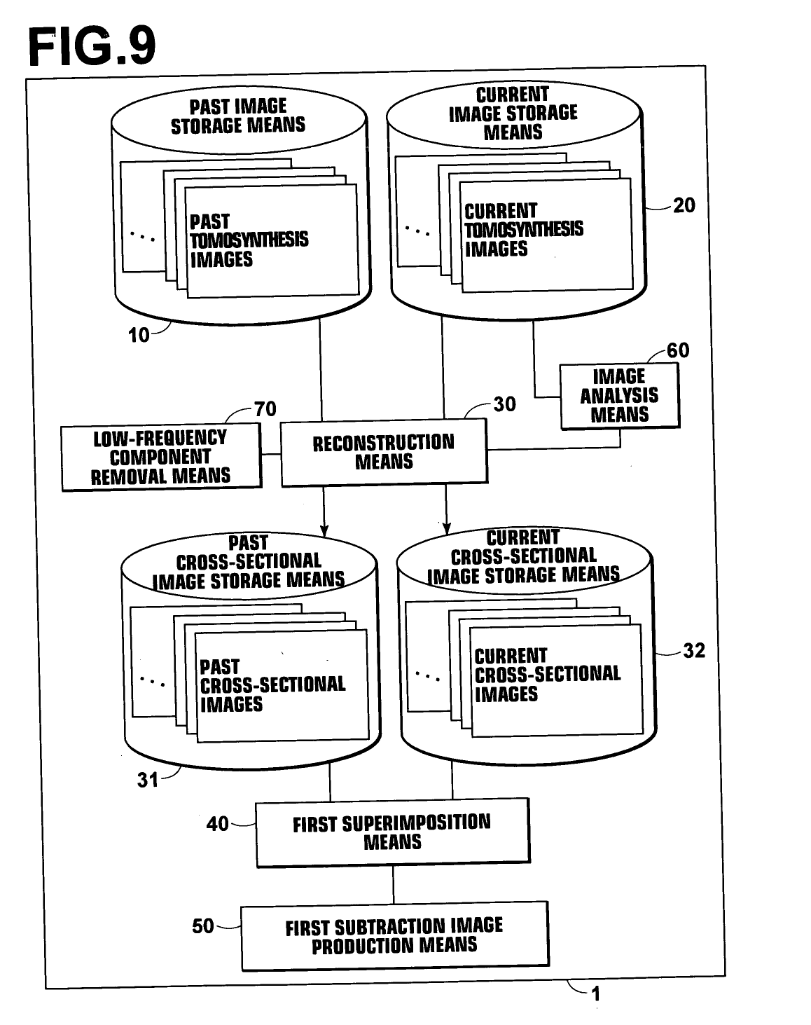

Further, as illustrated in FIG. 9, a low-frequency component removal means 70 maybe provided in the subtraction image production apparatus 1. The low-frequency component removal means 70 removes low-frequency components from each of the cross-sectional images reconstructed from the tomosynthesis images.

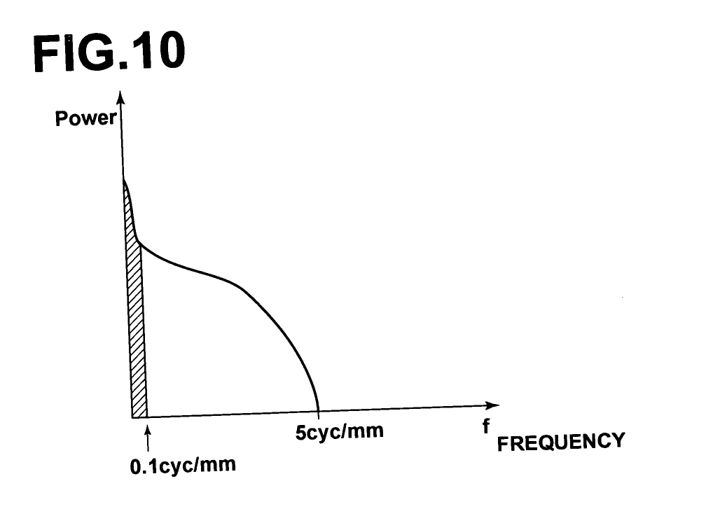

When the cross-sectional images are produced by moving the tomosynthesis images by a predetermined amount and adding the tomosynthesis images, noise may be generated in a low-frequency band during production of the cross-sectional images. If the cross-sectional images which were reconstructed from the tomosynthesis images by the reconstruction means 30 are decomposed into frequency components by fast Fourier transformation (FFT) or the like, a power value sharply rises at certain frequencies (shaded area) in the low-frequency band (0.1 cyc/mm or lower) as illustrated in FIG. 10, for example. Therefore, if the low-frequency components are removed by the low-frequency component removal means 70 by using a high-pass filter (or a low-pass filter) based on the characteristic of each of the cross-sectional images, a better observation result may be achieved. The high-pass filter is a filter which passes frequency components of which the frequencies are higher or equal to a predetermined frequency.

As described above in detail, since the subtraction images between the cross-sectional images are produced, it is possible to know on which cross-sectional plane the lesion has changed. Further, the conventional two-dimensional ordinary radiographic image includes information on each of the cross-sectional planes. Therefore, if there is a change in the posture of the subject, even if a subtraction image is produced, a difference between information which accurately corresponds to each other may not be obtained. In the subtraction image, the influence from the change in the posture is large. However, if a subtraction image is produced by aligning the structural elements in each cross-sectional image reconstructed from the tomosynthesis images as described above, the influence from the change in posture can be reduced. Accordingly, the radiologists can specify a cross-sectional plane on which the lesion has changed.

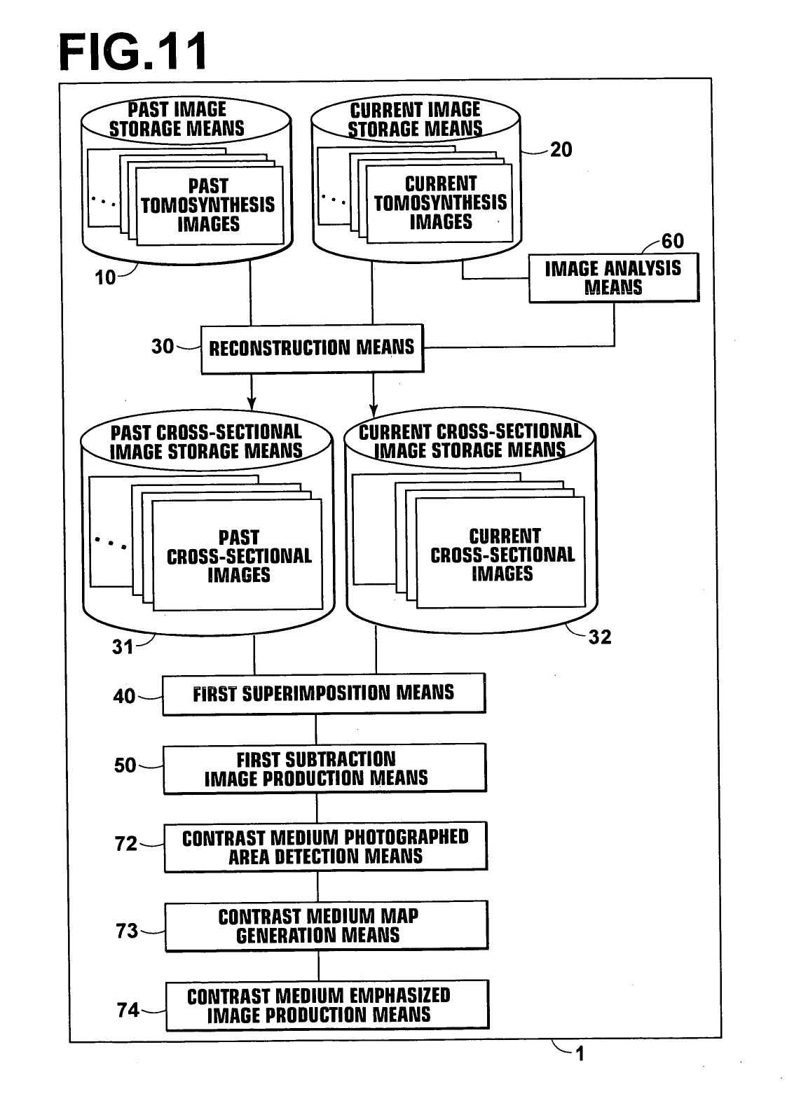

Further, images which were obtained by radiographing a subject before dosing the subject with a contrast medium are used as the past tomosynthesis images. Images which were obtained by radiographing the subject after dosing the subject with the contrast medium are used as the current tomosynthesis images. As illustrated in FIG. 11, a contrast medium photographed area detection means 72 for detecting a contrast medium photographed area in a subtraction image may be provided in the subtraction image production apparatus 1. A contrast medium map generation means 73 for generating a contrast medium distribution map by accumulating a contrast medium photographed area obtained from each of the subtraction images may be also provided in the subtraction image production apparatus 1. A contrast medium distribution emphasized image production means 74 for producing a contrast medium distribution emphasized image may be provided in the subtraction image production apparatus 1. The contrast medium distribution emphasized image is an image which is produced by superimposing the contrast medium distribution map produced by the contrast medium map generation means 73 on one of the plurality of tomosynthesis images obtained by radiographing the subject.

When a subject is photographed by tomosynthesis, a long time is normally required. Therefore, if a contrast medium which concentrates at cancers and arteriosclerotic lesions is used, the contrast medium is accumulated in a target organ of photography for a predetermined time. Therefore, it is preferable to use such a contrast medium in the present embodiment. In this case, the contrast medium is not photographed in the past tomosynthesis images. The contrast medium is photographed only in the current tomosynthesis images, and the contrast medium appears as white parts in the current tomosynthesis images. Therefore, the contrast medium appears as white parts in cross-sectional images which are reconstructed from the current tomosynthesis images. However, the contrast medium does not appear in cross-sectional images which are produced by reconstructing the past tomosynthesis images. Therefore, when a subtraction image is produced by subtracting the past cross-sectional image from the current cross-sectional image, structural elements such as a lung and ribs are removed, and only the area in which the contrast medium was photographed remains as a white area.

Therefore, the contrast medium photographed area detection means 72 detects an area which includes pixels having pixel values of 50 through 100, based on the pixels in the subtraction image, as the contrast medium photographed area in an image represented in 12 bits (four digits), for example.

Further, the contrast medium generation means 73 superimposes a contrast medium photographed area obtained from each subtraction image as well as the contrast medium photographed area of each cross-sectional image. Accordingly, the contrast medium generation means 73 generates a contrast medium distribution map in which the contrast medium is distributed to the entire photographed area.

Further, the contrast medium distribution emphasized image production means 74 may produce a contrast medium distribution emphasized image by superimposing the contrast medium distribution map generated by the contrast medium map generation means 73 on one of the past tomosynthesis images. If the contrast medium distribution emphasized image is produced, the photographed area of the contrast medium can be easily compared with the photographed area of the structural elements of the subject.

If a difference between the cross-sectional images which are reconstructed from the tomosynthesis images is obtained, even if the density of the contrast medium is low, the area in which the contrast medium was photographed is emphasized. Further, since the subtraction image is obtained, structural elements surrounding the contrast medium can be removed. Hence, the area in which the contrast medium was photographed is emphasized.

Further, a light pattern, such as that indicative of lung cancer, is not detected in some cases because of artifacts (Motion Artifact) created by cardiac cycles or respiration. Therefore, it is preferable to photograph the subject to obtain the current tomosynthesis images so that the phase of the cardiac cycle of the current tomosynthesis images is synchronized with that of the past tomosynthesis images. For that purpose, it is preferable to connect a cardiac cycle phase detector or a respiration phase detector to a photography device so that the current tomosynthesis images are synchronized with the past tomosynthesis images. In this case, the current tomosynthesis images are photographed so that the phases of the plurality of current tomosynthesis images are the same.

Specifically, a cardiograph, sphygmograph, or the like for detecting the phase of the cardiac cycle of the subject may be used as the cardiac cycle phase detector which will be connected to the photography device. When the subject is photographed to obtain the current tomosynthesis images, the cardiac cycle phase detector as described above is attached to the subject. The cardiac cycle phase detector detects the phase of the cardiac cycle based on the contraction and dilatation of the heart as analog signals. Then, the detected analog signals are converted into digital signals by A/D conversion. Accordingly, the signals are transmitted to the photography device in real time. When the phase of the cardiac cycle of the subject becomes the same as the phase of the cardiac cycle in the past, the subject is photographed by tomosynthesis at each position S1, S2, . . . and Sn. Further, the data on the cardiac cycle when the subject was photographed in the past should be stored in the photography device by attaching the data to the past tomosynthesis images (the plurality of past tomosynthesis images obtained when the phase of the cardiac cycle was the same).

Similarly, the current image is obtained by radiographing the subject so that the phase of respiration of the current image is the same as that of the past image. Specifically, a spirometer, respiration monitor belt, or optical camera is used to observe the movement of the patient's chest to monitor the respiration of the patient. Then, detected analog signals are converted into digital signals by A/D conversion and the signals are transmitted to the photography device in real time. Alternatively, instead of using the respiration phase detector, the photography device may scan the chest by irradiating it with a low dose to obtain an X-ray image of the chest. Then, the X-ray image of the chest may be evaluated in real time to obtain the phase of respiration. Then, the obtained phase of respiration may be sent to the photography device.

Alternatively, both of the cardiac cycle and the respiration may be monitored. Current tomosynthesis images and past tomosynthesis images in which both the cardiac cycle and the respiration are synchronized with each other may be used.

As described above, since the current tomosynthesis images and the past tomosynthesis images in which the cardiac cycle and the respiration are synchronized with each other are used, it is possible to observe a pattern even if the pattern is a light pattern such as that indicative of lung cancer.

Next, a second embodiment of the present invention will be described. In the second embodiment, tomosynthesis images which were obtained by radiographing a subject in the past by tomosynthesis are used as the past images, and an ordinary radiographic image obtained by ordinary photography is used as a current image. The same reference numerals are assigned to the same elements as those of the first embodiment, and detailed descriptions on them will be omitted.

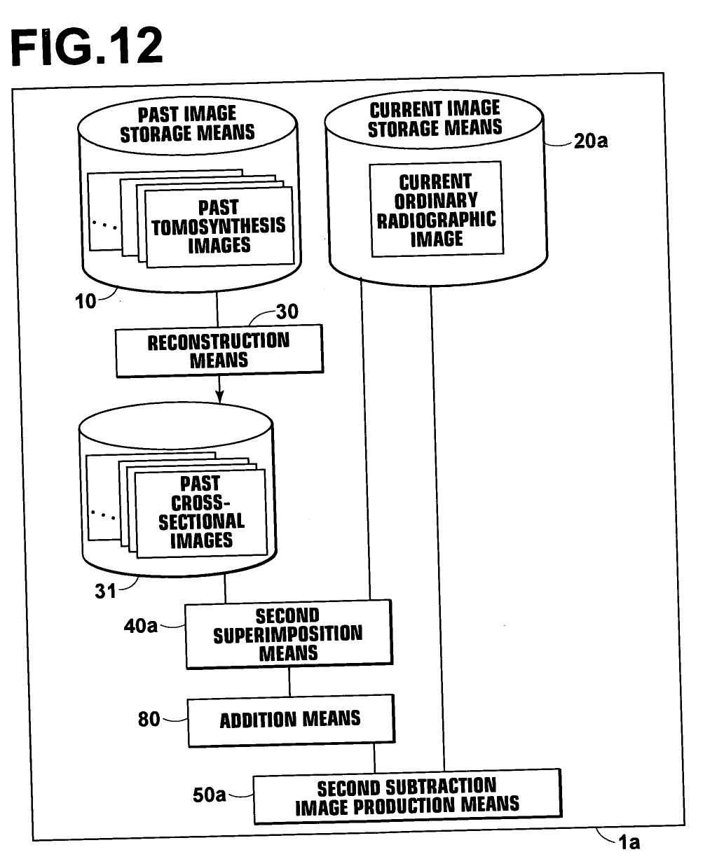

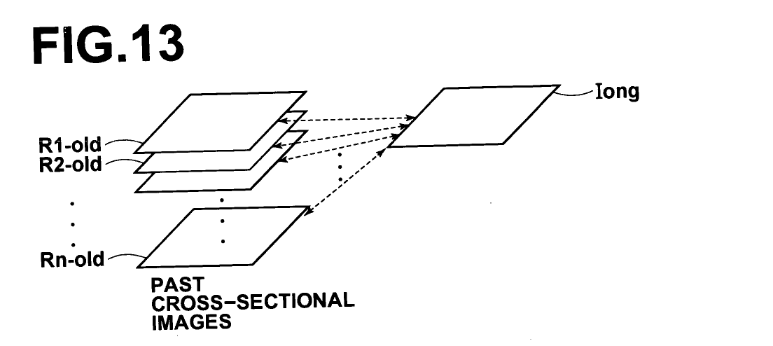

As illustrated in FIG. 12, A subtraction image production apparatus 1a according to the present invention includes a past image storage means 10. The past image storage means 10 stores past tomosynthesis images obtained in the past by radiographing a subject by tomosynthesis. The subtraction image production apparatus 1a also includes a current image storage means 20a. The current image storage means 20a stores a current ordinary radiographic image obtained by radiographing the subject by ordinary photography. The subtraction image production apparatus 1a also includes a reconstruction means 30 for reconstructing cross-sectional images from the tomosynthesis images. The subtraction image production apparatus 1a also includes a second superimposition means 40a for superimposing past cross-sectional images which were reconstructed from the past tomosynthesis images on the current ordinary radiographic image. The subtraction image production apparatus 1a also includes an addition image production means 80 for producing an addition image by adding all of the cross-sectional images, among which the structural elements have been aligned. The subtraction image production apparatus 1a also includes a second subtraction image production means 50a for producing a subtraction image between the addition image and the ordinary radiographic image.

The second superimposition means 40a superimposes the cross-sectional images reconstructed from the past tomosynthesis images on the ordinary radiographic image so as to align the structural elements therein by using various methods. For example, the methods which were described to explain the first superimposition means may be used.

Here, a flow of observation of a temporal change in a predetermined region of a subject using the subtraction image apparatus according to the present invention will be specifically described. Cross-sectional images reconstructed from the tomosynthesis image are used as the past images. An ordinary radiographic image is used as the current image.

First, while an X-ray tube of an X-ray photography device is moved sequentially from point S1 to S2, . . . , and Sn, the subject is photographed by tomosynthesis. A plurality of tomosynthesis images is obtained by photography, and stored in the past image storage means 10.

After a predetermined time has passed, the same region of the same subject is photographed again by ordinary photography to observe a temporal change in a lesion of the subject. The obtained ordinary radiographic image is stored in the current image storage means 20a.

The reconstruction means 30 produces cross-sectional images from the tomosynthesis images stored in the past image storage means 10. The cross-sectional images are produced at an interval of approximately 5 mm through 1 cm, for example. Cross-sectional images R1-old, R2-old, . . . , and Rn-old are reconstructed from the past tomosynthesis images, and stored in the past cross-sectional image storage means 31.

Then, the second superimposition means 40a superimposes past cross-sectional images R1-old, R2-old, . . . , and Rn-old, which are stored in the past cross-sectional image storage means 31, on an ordinary radiographic image Iorg, which is stored in the current cross-sectional image storage means 20, so as to align structural elements therein (for example, R1-old and Iorg, R2-old and Iorg, . . . and Rn-old and Iorg).

Further, the addition image production means 80 produced an addition image (for example, a linear sum is obtained by giving a predetermined weight to each image). The addition image is obtained by adding all the past cross-sectional images R1-old, R2-old, . . . , and Rn-old, in which the structural elements have been aligned. The addition image is an image produced by superimposing the past cross-sectional images on the ordinary radiographic image Iorg so as to align the structural elements. Therefore, the second subtraction image production means 50a subtracts the ordinary radiographic image Iorg from the addition image to produce a subtraction image.

In the present embodiment, tomosynthesis images obtained by radiographing a subject by tomosynthesis are used as the past images. An ordinary radiographic image obtained by radiographing the subject by ordinary photography is used as the current image. However, the past image may be an image obtained by radiographing the subject by ordinary photography, and the current images may be images obtained by radiographing the subject by tomosynthesis.

Further, an image analysis means may be provided in a similar manner to the first embodiment. The image analysis means may perform image analysis on the current ordinary radiographic image stored in the current image storage means 20a. If a lesion candidate is detected as the result of image analysis, cross-sectional images are reconstructed from the past tomosynthesis image at narrower intervals, for example. The second subtraction image production means 50a produces a subtraction image by using the past cross-sectional images reconstructed from the past tomosynthesis image at narrower intervals. Accordingly, the radiologists can observe the image in detail.

Further, a low-frequency component removal means may be provided in a similar manner to the first embodiment. The low-frequency component removal means is a means for removing the low-frequency components in each of the cross-sectional images. The low-frequency component removal means can remove low-frequency noise which is generated when the cross-sectional images are reconstructed from the tomosynthesis images.

The conventional two-dimensional ordinary radiographic image includes information on each cross-section plane. When there is a change in the posture of the subject, even if a subtraction image is obtained, an accurate difference can not be observed. Therefore, an influence from the change in the posture is large in the subtraction image. However, if a subtraction image is obtained by superimposing each cross-sectional image reconstructed from the tomosynthesis image on the two-dimensional ordinary radiographic image so as to align the structural elements therein as described above, the influence from the change in the posture may be reduced.

If tomosynthesis images obtained before dosing the subject with a contrast medium are used as the past tomosynthesis images and tomosynthesis images obtained after dosing the subject with the contrast medium are used as the current tomosynthesis images in a similar manner to the first embodiment, a contrast medium photographed area can be observed in the subtraction image. The contrast medium photographed area is an area in which the contrast medium was photographed.

Further, the contrast medium photographed area detection means as described above may be provided. The contrast medium photographed area detection means can automatically detect a contrast medium photographed area in the subtraction image. Further, a contrast medium map generation means may be provided to generate a contrast medium map, and a contrast medium distribution emphasized image production means may be provided to produce a contrast medium emphasized image.

Further, it is preferable to obtain the current tomosynthesis images or current ordinary radiographic image by radiographing the subject so that the phase of the cardiac cycle of the current image or images is synchronized with that of the past image or images. For that purpose, it is preferable to use tomosynthesis images and an ordinary radiographic image which are obtained by radiographing the subject by connecting a cardiac cycle phase detector or a respiration phase detector to a photography device so that the phase of the cardiac cycle of the current image or images is synchronized with that of the past image or images.

In the first and second embodiments, a CR (computed radiography) apparatus is used as the X-ray photography device to photograph the subject. However, a detector such as a FPD (flat panel detector), which can handle dynamic images, may also be used. If the FPD is used, it is possible to improve a throughput of photography.

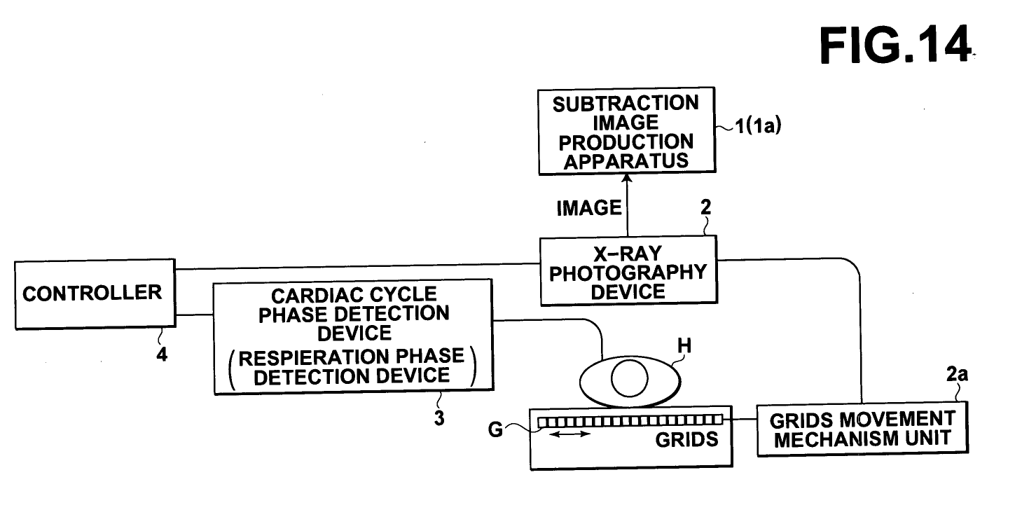

Further, in the first and second embodiments as described above, when a subject H is photographed with an X-ray photography device 2 as illustrated in FIG. 14, scattered ray elimination grids G may be provided between the subject H and a detection surface such as an imaging plate to prevent scattered rays from entering the detection surface. As a typical example of the scattered ray elimination grids G, there are scattered ray elimination grids formed at certain intervals by laminating a multiplicity of lead foils in parallel with the radiations from the X-ray radiation source. The lead is a radiation shielding material. However, when the subject is photographed using the scattered ray elimination grids G as described above, an image in which the structure of the scattered ray elimination grids G is superimposed on the image of the subject is produced.

Therefore, a grid moving mechanism unit 2a for moving the scattered radiation elimination grids G back and forth is provided in the X-ray photography device 2. The scattered radiation elimination grids G are moved in a direction crossing the laminated structure during exposure to the X-rays to blur the image of the grids. The image of the grids blurs more as the scattered radiation elimination grids G is moved more during exposure to the X-rays. Therefore, it is preferable that the scattered radiation elimination grids G are moved so that the moving speed of the scattered radiation elimination grids G is the maximum during exposure to the X-rays. Therefore, a controller 4 controls the movement of the grids movement mechanism unit 2a, based on the timing of sending an X-ray generation signal to the X-ray photography device 2, so that the scattered radiation elimination grids G move fastest during exposure to the X-rays.

Further, the openings of the scattered radiation elimination grids G are filled with a radiation permeable material such as wood and aluminum to support the physical structure of the grids. The grids movement mechanism unit 2a mechanically moves the scattered radiation elimination grids G back and forth. Therefore, the moving speed of the scattered radiation elimination grids G is not the fastest at the turning points of the movement. Therefore, the controller 4 should control the movement of the grids movement mechanism unit 2a so that they move fastest at the same timing as the phase of cardiac cycle (or the phase of respiration) at which the subject is photographed. For example, the controller 4 controls the movement of the grids movement mechanism unit 2a to move the scattered radiation elimination grids G so that the movement of the scattered radiation elimination grids G is synchronized with the phase of the cardiac cycle detected by the cardiac cycle phase detection device 3 (or the phase of respiration detected by the respiration phase detection device).

Further, if the subject is photographed without moving the scattered radiation elimination grids G, the laminated structure of the grids is superimposed on the image of the subject. Therefore, GPR processing (grids elimination processing) may be performed on the past images and the current images before the past images and the current images are input to the superimposition means and the subtraction image production means as described above.

Alternatively, it is also possible to photograph the subject without moving the scattered radiation elimination grids. The subject may be photographed by increasing the density of the grid layers so that the grids become unnoticeable. Generally, when the chest or the like of a subject is photographed, it is preferable to use grids of approximately 5 cyc/mm or higher (higher than or equal to a Nyquist frequency based on readout pixel density). When the breast of the subject is photographed, it is preferable to use grids of approximately 10 cyc/mm or higher (higher than or equal to a Nyquist frequency based on readout pixel density). High-density grids of which the density is less than or equal to a minimum resolution that can be detected by the detection unit should be used. The detection unit is a unit for detecting X-ray information accumulated on a detection plane such as an imaging plate. In other words, the density of the high-density grids should be less than or equal to the resolution of the current image or images.

Alternatively, instead of using the scattered radiation elimination grids, a so-called Groedel method may be used. The Groedel method is a method for eliminating scattered radiation from the subject by placing the subject and the detection plane, such as the imaging plate, apart from each other by 15 to 20 cm.

As described above, an image which is not influenced by the scattered radiation can be obtained by using the scattered radiation elimination grids or by placing the subject and the detection plane apart from each other by a certain distance.

Further, a program for executing each of the means described in each of the above embodiments on a computer may be recorded on a recording medium such as a CD-ROM, and installed in a computer. Further, the program may be sent via a network and installed in a computer to cause the computer to operate as a subtraction image production apparatus.

1. A subtraction image production apparatus comprising:
a superimposition means for superimposing a past cross-sectional image on a corresponding current cross-sectional image for each cross-sectional plane so as to align structural elements therein, when there are a plurality of past cross-sectional images, which have been reconstructed from a plurality of past tomosynthesis images obtained by radiographing a subject by tomosynthesis, and a plurality of current cross-sectional images, which have been reconstructed from a plurality of current tomosynthesis images obtained by radiographing the subject by tomosynthesis; and
a subtraction image production means for producing a subtraction image between each pair of the past cross-sectional image and the current cross-sectional image, in which the structural elements have been aligned.

2. A subtraction image production apparatus as defined in claim 1, further comprising:
an image analysis means for performing image analysis on at least one of the current tomosynthesis images and the current cross-sectional images; and
a reconstruction means for reconstructing the plurality of past cross-sectional images from the plurality of past tomosynthesis images at a narrower interval and reconstructing the plurality of current cross-sectional images from the plurality of current tomosynthesis images at a narrower interval if the image analysis means finds a lesion candidate.

3. A subtraction image production apparatus as defined in claim 1, further comprising:
a low-frequency component removal means for removing a low-frequency component from each of the reconstructed cross-sectional images.

4. A subtraction image production apparatus as defined in claim 1, wherein the phase of the cardiac cycle and/or the phase of respiration of the plurality of past tomosynthesis images is the same, and wherein the subject is photographed so that the phase of the cardiac cycle and/or the phase of respiration of the plurality of current tomosynthesis images is the same as that of the plurality of past tomosynthesis images.

5. A subtraction image production apparatus as defined in claim 4, wherein the plurality of past tomosynthesis images was obtained by radiographing the subject before the subject was dosed with a contrast medium, and wherein the plurality of current tomosynthesis images was obtained by radiographing the subject after the subject was dosed with the contrast medium.

6. A subtraction image production apparatus as defined in claim 5, further comprising:
a contrast medium photographed-area detection means for detecting the photographed area of the contrast medium including pixels of which the pixel values are greater than or equal to a predetermined pixel value in each of the subtraction images.

7. A subtraction image production apparatus as defined in claim 6, further comprising:
a contrast medium map generation means for generating a contrast medium distribution map by superimposing the photographed area of the contrast medium, obtained from each of the subtraction images, on each other.

8. A subtraction image production apparatus as defined in claim 7, further comprising:
a contrast medium distribution emphasized image production means for producing a contrast medium distribution emphasized image by superimposing the contrast medium distribution map, generated by the contrast medium map generation means, on at least one of the plurality of past tomosynthesis images.

9. A subtraction image production apparatus comprising:
a superimposition means for superimposing each cross-sectional image reconstructed from a plurality of tomosynthesis images obtained by radiographing a subject by tomosynthesis on an ordinary radiographic image obtained by radiographing the subject by ordinary radiographic photography so as to align structural elements therein, when there are the ordinary radiographic image and a plurality of cross-sectional images reconstructed from the plurality of tomosynthesis images, and one of the ordinary radiographic image and the plurality of tomosynthesis images is a past image or past images and the other is a current image or current images;
an addition image production means for producing an addition image by adding all of the cross-sectional images, in which the structural elements have been aligned; and
a subtraction image production means for producing a subtraction image between the addition image and the ordinary radiographic image.

10. A subtraction image production apparatus as defined in claim 9, wherein the past ordinary radiographic image or the plurality of past tomosynthesis images was obtained by radiographing the subject before the subject was dosed with a contrast medium, and wherein the current ordinary radiographic image or the plurality of current tomosynthesis images was obtained by radiographing the subject after the subject was dosed with the contrast medium.

11. A subtraction image production apparatus as defined in claim 9, wherein the phase of the cardiac cycle and/or the phase of respiration of the plurality of past tomosynthesis images is the same, and wherein the current ordinary radiographic image was obtained by radiographing the subject so that the phase of the cardiac cycle and/or the phase of respiration of the current ordinary radiographic image is the same as that of the plurality of past tomosynthesis images.

12. A subtraction image production apparatus as defined in claim 9, wherein the plurality of current tomosynthesis images was obtained by radiographing the subject so that the phase of the cardiac cycle and/or the phase of respiration of the plurality of current tomosynthesis images is the same as that of the past ordinary radiographic image.

13. A subtraction image production method comprising the steps of:
superimposing a past cross-sectional image on a corresponding current cross-sectional image for each cross-sectional plane, when there are a plurality of past cross-sectional images, which have been reconstructed from a plurality of past tomosynthesis images obtained by radiographing a subject by tomosynthesis, and a plurality of current cross-sectional images reconstructed from a plurality of current tomosynthesis images obtained by radiographing the subject by tomosynthesis; and
producing a subtraction image between each pair of the past cross-sectional image and the current cross-sectional image, in which the structural elements have been aligned.

14. A subtraction image production method comprising the steps of:
superimposing each cross-sectional image reconstructed from a plurality of tomosynthesis images obtained by radiographing a subject by tomosynthesis on an ordinary radiographic image obtained by radiographing the subject by ordinary radiographic photography so as to align structural elements therein, when there are the ordinary radiographic image and a plurality of cross-sectional images reconstructed from the plurality of tomosynthesis images, and one of the ordinary radiographic image and the plurality of tomosynthesis images is a past image or past images and the other is a current image or current images;
producing an addition image by adding all of the cross-sectional images in which the structural elements have been aligned; and
producing a subtraction image between the addition image and the ordinary radiographic image.

15. A program for causing a computer to execute a subtraction image production method, the program comprising the procedures for:
superimposing a past cross-sectional image on a corresponding current cross-sectional image for each cross-sectional plane so as to align structural elements therein, when there are a plurality of past cross-sectional images, which have been reconstructed from a plurality of past tomosynthesis images obtained by radiographing a subject by tomosynthesis, and a plurality of current cross-sectional images, which have been reconstructed from a plurality of current tomosynthesis images obtained by radiographing the subject by tomosynthesis; and
producing a subtraction image between each pair of the past cross-sectional image and the current cross-sectional image, in which the structural elements have been aligned.

16. A program for causing a computer to execute a subtraction image production method, the program comprising the procedures for:
superimposing a cross-sectional image reconstructed from a plurality of tomosynthesis images obtained by radiographing a subject by tomosynthesis on an ordinary radiographic image obtained by radiographing the subject by ordinary radiographic photography so as to align structural elements therein, when there are the ordinary radiographic image and a plurality of cross-sectional images reconstructed from the plurality of tomosynthesis images, and one of the ordinary radiographic image and the plurality of tomosynthesis images is a past image or past images and the other one is a current image or current images;
producing an addition image by adding all of the cross-sectional images in which the structural elements have been aligned; and
producing a subtraction image between the addition image and the ordinary radiographic image.
